(19) United States
(12) Patent Application Publication
(10) Pub. No.: US 2023/0005616 A1
(43) Pub. Date: Jan. 5, 2023

(54) METHODS, SYSTEMS, AND COMPUTER PROGRAM PRODUCTS FOR CAPTURING MISSING CURRENT PROCEDURAL TERMINOLOGY (CPT) CODES FOR CARE PROVIDED TO A PATIENT

(71) Applicant: Change Healthcare Holdings LLC, Nashville, TN (US)

(72) Inventors: Wenji Zhang, Redmond, WA (US); Feili Yu, Shoreline, WA (US); Kaushik Roy, Foster City, CA (US)

(21) Appl. No.: 17/364,611

(22) Filed: Jun. 30, 2021

(51) Int. Cl. G16H 50/20; G16H 70/20; G16H 40/20; G16H 10/60; G06Q 40/08; G16H 50/70; G06N 5/02; G06N 20/00

(52) U.S. Cl. CPC G16H 50/20; G16H 70/20; G16H 40/20; G16H 10/60; G06Q 40/08; G16H 50/70; G06N 5/027; G06N 20/00; G06Q 50/22

(57) ABSTRACT

A method includes receiving a current claim associated with care provided to a patient by a provider, the claim including a current diagnosis code and a first current procedural terminology (CPT) code that are based on the care that was provided; identifying, using an artificial intelligence (AI) engine, a second current CPT code based on the current diagnosis code and the first current CPT code; and generating a current confidence level value that the second current CPT code is missing from the current claim.

### FIELD

The present inventive concepts relate generally to health care systems and services and, more particularly, to the use of artificial intelligence (AI) in managing patient care.

### BACKGROUND

In caring for a patient, a health care service provider may interact with one or more payors, such as insurance companies, government agencies, and the like, that are responsible for paying for all or a portion of the patient's expenses. A health care service provider may generate claims for the paying entity that each may include a diagnosis code and one or more current procedural terminology (CPT) codes. CPT codes are numbers assigned to every task and service a health care service provider may provide to a patient including, but not limited to, medical, surgical, and diagnostic services. They are used by payors to determine the amount of reimbursement that a health care service provider will receive by a payor for that service. When compiling a claim for submission to a payor, however, a health care service provider may miss one or more codes for services or products rendered to the patient. This may be the result of an oversight by an individual responsible for entering the code where the code is never entered, an entry mistake where the code is entered incorrectly, a naming convention where a health care service provider uses a different code to represent a group of CPT codes, or other reason that results in a claim being generated that is missing one or more CPT codes for services or products rendered to the patient. These missing CPT codes may result in a health care service provider being inadequately reimbursed for services or products rendered and/or additional expenses in submitting amended or revised claims to payors.

### SUMMARY

According to some embodiments of the inventive concept, a method comprises receiving a current claim associated with care provided to a patient by a provider, the claim including a current diagnosis code and a first current procedural terminology (CPT) code that are based on the care that was provided; identifying, using an artificial intelligence (AI) engine, a second current CPT code based on the current diagnosis code and the first current CPT code; and generating a current confidence level value that the second current CPT code is missing from the current claim.

In other embodiments, the method further comprises generating a notification that the second current CPT code is missing from the current claim when the confidence level value exceeds a threshold value.

In still other embodiments, identifying, using the (AI) engine, the second current CPT code based on the current diagnosis code and the first current CPT code comprises: identifying, using an association rules learning (ARL) engine, the second current CPT code based on the current diagnosis code and the first current CPT code.

In still other embodiments, the method further comprises training the ARL engine using a plurality of historical claims associated with past care provided to a plurality of patients, the plurality of historical claims containing a plurality of diagnosis codes and a plurality of CPT codes.

In still other embodiments, training the ARL engine comprises: identifying, for each of the plurality of diagnosis codes, if condition-then result associations between ones of the plurality of CPT codes.

In still other embodiments, the method further comprises generating, for each of the plurality of diagnosis codes, at least one rule based on the if condition-then result associations between ones of the plurality of CPT codes. Each of the at least one rule comprises an antecedent corresponding to the if condition, a consequent corresponding to the then result, a rule confidence level value that the consequent is found in combination with the antecedent in the plurality of historical claims for the respective one of the plurality of diagnosis codes.

In still other embodiments, the method further comprises determining a rule support value for each of the plurality of CPT codes in the plurality of historical claims based on a frequency of occurrence in the plurality of historical claims for each of the plurality of CPT codes; determining a confidence level value for each of the if condition-then result associations between ones of the plurality of CPT codes based on a proportion of a number of times each of the at least one rule is found to be true relative to a frequency of a number of occurrences of the antecedent; and selecting, for each of the plurality of diagnosis codes, the at least one rule based on the rule support values and the confidence level values.

In still other embodiments, generating the current confidence level value that the second current CPT code is missing from the current claim comprises: generating the current confidence value level as the rule confidence level value for one of the at least one rule corresponding to the current diagnosis code having the first CPT code as the antecedent and the second CPT code as the consequent.

In still other embodiments, one of the at least one rule comprises one or more of the plurality of CPT codes as the antecedent and the one or more of the plurality of CPT codes as the consequent.

In still other embodiments, the current claim includes a third CTP code; identifying, using the AI engine, the second CPT code comprises identifying, using the AI engine, the second CPT code based on the current diagnosis code, the first CPT code, and the third CPT code; and generating the current confidence level value comprises generating the current confidence value level as the rule confidence level value for one of the at least one rule corresponding to the current diagnosis code having the first CPT code and the third CPT code as the antecedent and the second CPT code as the consequent.

In still other embodiments, identifying, using the AI engine, the second CPT code comprises identifying, using the AI engine, the second CPT code and a third CPT code based on the current diagnosis code and the first CPT code; and generating the current confidence level value comprises generating the current confidence value level as the rule confidence level value for one of the at least one rule corresponding to the current diagnosis code having the first CPT code as the antecedent and the second CPT code and the third CPT code as the consequent.

In still other embodiments, the current claim includes a third CTP code; identifying, using the AI engine, the second CPT code comprises identifying, using the AI engine, the second CPT code and a fourth CPT code based on the current diagnosis code, the first CPT code, and the third CPT code; and generating the current confidence level value comprises generating the current confidence value level as the rule confidence level value for one of the at least one rule corresponding to the current diagnosis code having the first CPT code and the third CPT code as the antecedent and the second CPT code and the fourth CPT code as the consequent.

According to some embodiments of the inventive concept, a system comprises a processor; and a memory coupled to the processor and comprising computer readable program code embodied in the memory that is executable by the processor to perform operations comprising: receiving a current claim associated with care provided to a patient by a provider, the claim including a current diagnosis code and a first current procedural terminology (CPT) code that are based on the care that was provided; identifying, using an artificial intelligence (AI) engine, a second current CPT code based on the current diagnosis code and the first current CPT code; and generating a current confidence level value that the second current CPT code is missing from the current claim.

In further embodiments, identifying, using the (AI) engine, the second current CPT code based on the current diagnosis code and the first current CPT code comprises: identifying, using an association rules learning (ARL) engine, the second current CPT code based on the current diagnosis code and the first current CPT code.

In still further embodiments, the operations further comprise training the ARL engine using a plurality of historical claims associated with past care provided to a plurality of patients, the plurality of historical claims containing a plurality of diagnosis codes and a plurality of CPT codes.

In still further embodiments, training the ARL engine comprises: identifying, for each of the plurality of diagnosis codes, if condition-then result associations between ones of the plurality of CPT codes.

In still further embodiments, the operations further comprise generating, for each of the plurality of diagnosis codes, at least one rule based on the if condition-then result associations between ones of the plurality of CPT codes. Each of the at least one rule comprises an antecedent corresponding to the if condition, a consequent corresponding to the then result, a rule confidence level value that the consequent is found in combination with the antecedent in the plurality of historical claims for the respective one of the plurality of diagnosis codes.

In still further embodiments, the operations further comprise determining a rule support value for each of the plurality of CPT codes in the plurality of historical claims based on a frequency of occurrence in the plurality of historical claims for each of the plurality of CPT codes; determining a confidence level value for each of the if condition-then result associations between ones of the plurality of CPT codes based on a proportion of a number of times each of the at least one rule is found to be true relative to a frequency of a number of occurrences of the antecedent; and selecting, for each of the plurality of diagnosis codes, the at least one rule based on the rule support values and the confidence level values.

In some embodiments of the inventive concept, a computer program product comprises a non-transitory computer readable storage medium comprising computer readable program code embodied in the medium that is executable by a processor to perform operations comprising: receiving a current claim associated with care provided to a patient by a provider, the claim including a current diagnosis code and a first current procedural terminology (CPT) code that are based on the care that was provided; identifying, using an artificial intelligence (AI) engine, a second current CPT code based on the current diagnosis code and the first current CPT code; and generating a current confidence level value that the second current CPT code is missing from the current claim.

In other embodiments, identifying, using the (AI) engine, the second current CPT code based on the current diagnosis code and the first current CPT code comprises: identifying, using an association rules learning (ARL) engine, the second current CPT code based on the current diagnosis code and the first current CPT code.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a current procedural terminology (CPT) code capture system in accordance with some embodiments of the inventive concept;

FIG. 2 is a block diagram that illustrates the CPT code capture system of FIG. 1 in accordance with some embodiments of the inventive concept;

FIG. 3 is a block diagram that illustrates historical claim information used as an input to the CPT code capture system in accordance with some embodiments of the inventive concept;

FIG. 4 is a block diagram that illustrates tables that represent rules corresponding to if condition-then result associations between CPT codes in accordance with some embodiments of the inventive concept;

FIG. 5 is a graph that illustrates conditional probabilities that CPT codes are associated with one another for a particular diagnosis code in accordance with some embodiments of the inventive concept;

FIGS. 6-8 are flowcharts that illustrate operations for capturing missing CPT codes from a claim in accordance with some embodiments of the inventive concept;

FIG. 9 is a data processing system that may be used to implement a CPT code capture system in accordance with some embodiments of the inventive concept; and

FIG. 10 is a block diagram that illustrates a software/hardware architecture for use in in a CPT code capture system in accordance with some embodiments of the inventive concept.

### DETAILED DESCRIPTION

In the following detailed description, numerous specific details are set forth to provide a thorough understanding of embodiments of the inventive concept. However, it will be understood by those skilled in the art that embodiments of the inventive concept may be practiced without these specific details. In some instances, well-known methods, procedures, components and circuits have not been described in detail so as not to obscure the inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

Embodiments of the inventive concept are described herein in the context of a current procedural terminology (CPT) code capture system that includes an artificial intelligence (AI) engine, which uses machine learning and association rules learning (ARL). It will be understood that embodiments of the inventive concept are not limited to a machine learning and/or ARL implementation of the CPT code capture system and other types of AI systems may be used including, but not limited to, a multi-layer neural network, a deep learning system, a natural language processing system, and/or computer vision system. Moreover, it will be understood that the multi-layer neural network is a multi-layer artificial neural network comprising artificial neurons or nodes and does not include a biological neural network comprising real biological neurons.

Some embodiments of the inventive concept stem from a realization that when compiling a claim for submission to a payor, a provider may miss one or more CPT codes for services or products rendered to a patient. These missing codes may be identified manually by coders and/or reviewers, but this may be a slow, labor intensive process. Embodiments of the inventive concept may provide a CPT code capture system that includes an AI engine to analyze historical claim information to detect patterns between CPT codes contained therein to identify potential candidate CPT code(s) that may be missing from a current claim. In some embodiments, the AI engine may use an ARL engine to identify co-occurrences of CPT codes in historical claims. Rules may be derived based on these co-occurrences and, based on the frequency that the individual rules are found to be true in the historical claim information, confidence level values may be determined for the various rules. The rules may be organized, and tables generated according to diagnosis code. These tables may be applied to a current claim to determine the likelihood that one or more CPT codes are missing from the claim based on the CPT codes in the claim and the confidence levels contained in the tables for the various associations between CPT codes. For example, if a table has been generated for diagnosis code 14891, and the table expresses a rule that if CPT code 80053 is present in a claim, then there is about a 62% likelihood that CPT code 36415 is also present. If a claim includes CPT code 80053, but does not include CPT code 36415, then it may be beneficial to scrutinize the claim further to consider adding CPT code 36415 as CPT codes 80053 and 36415 occur together at a relatively high frequency. The CPT code capture system may, therefore, allow coders and/or reviewers to focus on those claims that are more likely to have coding errors therein, which may reduce the labor and time involved in manually reviewing claims for coding errors.

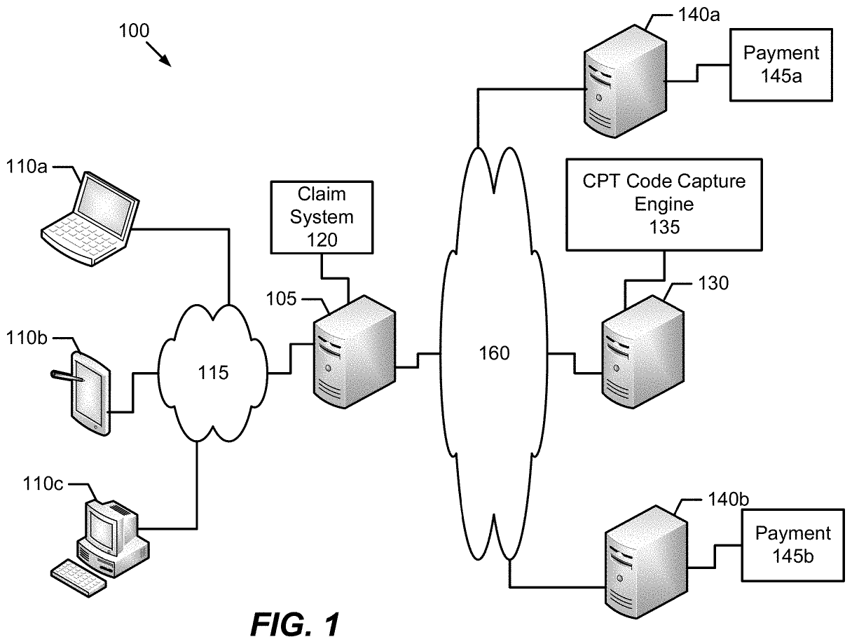

Referring to FIG. 1, a communication network 100 including a CPT code capture system, in accordance with some embodiments of the inventive concept, comprises a health care facility server 105 that is coupled to devices 110a, 110b, and 110c via a network 115. The health care facility may be any type of health care or medical facility, such as a hospital, doctor's office, specialty center (e.g., surgical center, orthopedic center, laboratory center etc.), or the like. The health care facility server 105 may be configured with a claim system module 120 to manage patient files and facilitate the compilation of patient care data. This patient care data may be generated from health care products and services provided to the patients through health care service providers. The providers may use devices, such as devices 110a, 110b, and 110c, to manage patients' electronic records and to issue orders for the patients. An order may include, but is not limited to, a treatment, a procedure (e.g., surgical procedure, physical therapy procedure, radiologic/imaging procedure, etc.) a test, a prescription, and the like. Thus, the claim system module 120 of the health care facility server 105 may be configured to process the data in the patients' electronic medical records and to compile this information into potential claims for payment from one or more entities on behalf of the individual patients. In the example shown in FIG. 1, two different entities are illustrated: a government entity, such as the Center for Medicare and Medicaid Services (CMS) is represented by a CMS server 140a, and an insurance entity is represented by an insurance server 140b. The CMS server 140a and the insurance server 140b have payment modules 145a and 145b, respectively, that are configured to process each of the claims submitted thereto for reimbursement. Each of the claims may be approved for reimbursement or denied in whole or in part due to one or more deficiencies. The network 115 communicatively couples the devices 110a, 110b, and 110c to the health care facility server 105. The network 115 may comprise one or more local or wireless networks to communicate with the health care facility server 105 when the health care facility server 105 is located in or proximate to the health care facility. When the health care facility server 105 is in a remote location from the health care facility, such as part of a cloud computing system or at a central computing center, then the network 115 may include one or more wide area or global networks, such as the Internet.

According to some embodiments of the inventive concept, a CPT code capture system including a CPT code capture server 130 and a CPT code capture engine module 135 may be used to analyze claims generated by one or more providers to identify potential missing CPT codes. The CPT code capture engine 135 may include an artificial intelligence (AI) engine to analyze historical claim information to detect patterns between CPT codes contained therein. In some embodiments, the AI engine may use an association rules learning (ARL) engine to identify co-occurrences of CPT codes in historical claims. Based on these patterns, if condition-then result rules may be generated in which the various CPT codes correspond to the antecedent (if condition) and the consequent (then condition). Each rule may have a confidence level value assigned thereto, which is a measure of the likelihood that the consequent is present in a claim, i.e., the rule is found to be true, when the antecedent is present in a claim. Thus, CPT codes associated with a particular diagnosis code in a claim may be used to search the antecedent field in the table for that particular diagnosis code. Potential missing CPT codes from the consequent fields in the table may be identified for each of the CPT codes currently in the claim. When the confidence level is above a defined threshold, a coder and/or reviewer may be notified to review the claim for consideration of adding one or more of the potential missing CPT codes or determining if there are other errors in the claim.

Although FIG. 1 illustrates an example communication network including a CPT code capture system, it will be understood that embodiments of the inventive concept are not limited to such configurations, but are intended to encompass any configuration capable of carrying out the operations described herein.

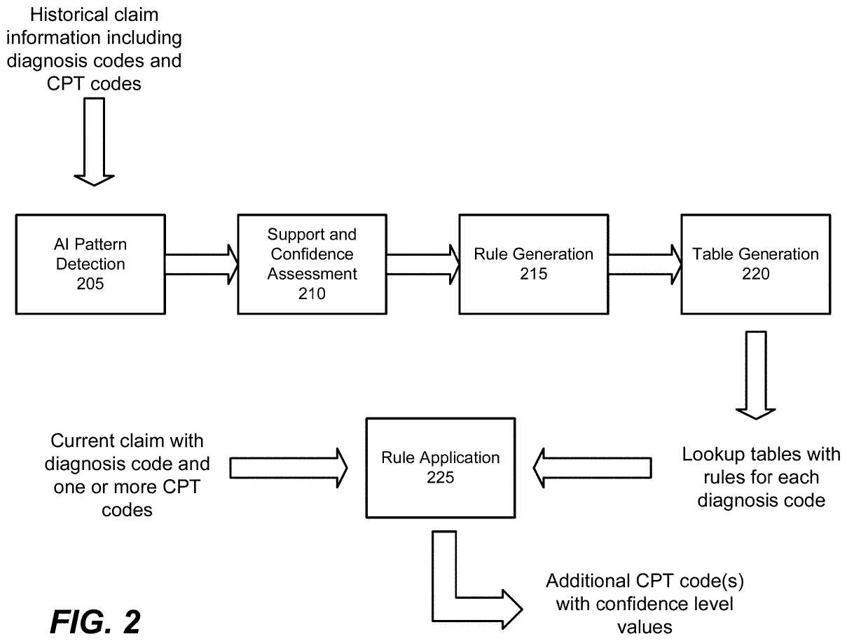

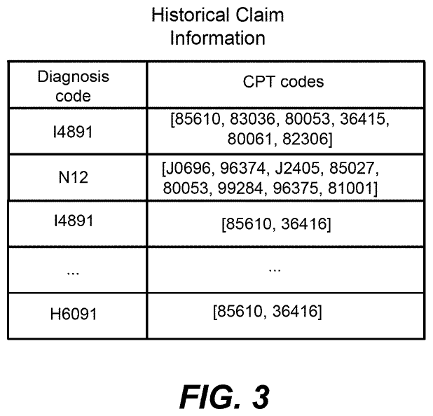

FIG. 2 is a block diagram that illustrates the CPT code capture system of FIG. 1 in accordance with some embodiments of the inventive concept. Referring to FIG. 2, historical claim information including diagnosis codes and CPT codes may be provided as input to an AI pattern detection module 205. FIG. 3 illustrates an example of the historical claim information organized by diagnosis code. As shown in FIG. 3, various diagnosis codes may have one or more CPT codes associated therewith. The AI pattern detection module 205 may be configured to use ARL to detect patterns in the CPT codes associated with each of the diagnosis codes contained in the historical claim information. ARL is a machine learning algorithm that can detect patterns or co-occurrences in the CPT codes associated with each of the diagnosis codes. These co-occurrences of the CPT codes may be expressed as if condition-then result associations from which rules may be derived. Depending on the size of the dataset there may be numerous potential rules that may be generate. In some embodiments, the more statistically significant rules may be selected from the universe of all possible rules base on the co-occurrences of the various CPT codes.

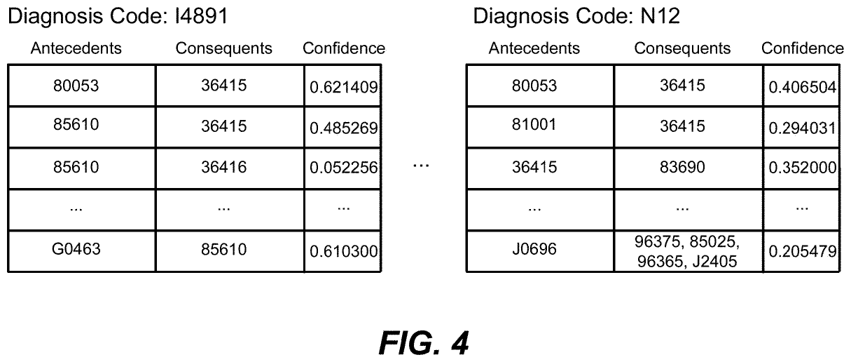

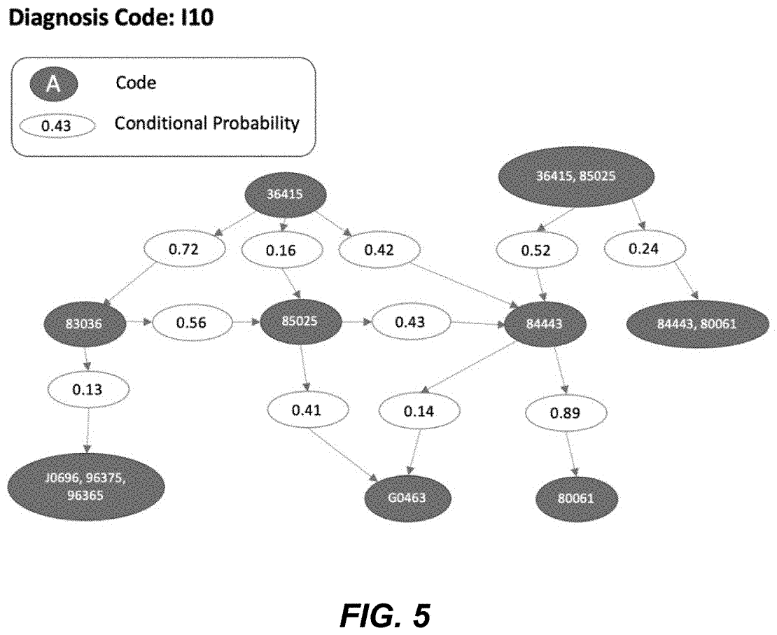

The support and confidence assessment module 210 may be configured to use support and confidence criteria to select rules for capturing potential missing CPT codes from claims in accordance with some embodiments of the inventive concept. The support criterion is a measure of how frequently each of the CPT codes or combination of the CPT codes, i.e., any set of CPT codes used as an antecedent or consequent, occurs in the historical claim information. The confidence criterion is an indication of the proportion of a number of times that a rule is true, i.e., occurrence of the consequent given the antecedent, to a number of occurrences of the antecedent. Based on these two criteria, the rule generation module 215 may be configured to generate one or more rules for each of the diagnosis codes. The table generation module 220 may be configured to express these rules in the form of tables where a table is created for each of the different diagnosis codes. FIG. 4 illustrates an example of these tables for two different diagnosis codes 14891 and N12. As shown in FIG. 4, the tables have CPT codes filled in for the various antecedents and consequents fields. Each row in the tables corresponds to a particular rule for that diagnosis code. The tables also include a confidence field that corresponds to the confidence criterion determined for that rule and the particular CPT codes associated therewith. As described above, any set of CPT codes may be used as an antecedent or consequent. Thus, the antecedent for each rule may be a single CPT code or multiple CPT codes and the consequent for each rule may be a single CPT code or multiple CPT codes in accordance with various embodiments of the inventive concept. The relationship between the various CPT codes and groups of CPT codes may be illustrated by way of a graph. FIG. 5 illustrates an example graph for a diagnosis code 110 in which the nodes represent various CPT codes and groups of CPT codes associated with the diagnosis code 110 from the historical claim information. The directional edges are weighted with their conditional probabilities or confidence levels indicating the likelihood that the CPT code(s) corresponding to the terminating node are present as a consequent when the CPT code(s) corresponding to the originating node are present as an antecedent.

Returning to FIG. 2, tables output from the table generation module 220 for each of the diagnosis codes in the historical claim information may be provided as input to a rule application module 225. The rule application module 225 may be configured to apply these lookup tables to a current claim with a particular diagnosis code and one or more CPT codes associated therewith. The CPT code(s) individually or in combination in the current claim may be used to determine if there is a match in the antecedent field for the lookup table associated with that diagnosis code. When a match is detected, the one or more CPT codes included in the consequent field may be identified along with the confidence value that is indicative of the likelihood that these CPT codes co-occur with the CPT code(s) in the antecedent field. When the confidence value exceeds a defined threshold and CPT code(s) in the consequent field are not present in the claim for this diagnosis code, then a coder or reviewer may be notified to manually review the claim for consideration of adding one or more of the potentially missing codes and/or correcting other errors in the claim that may be uncovered by this investigation into the potential missing CPT code(s).

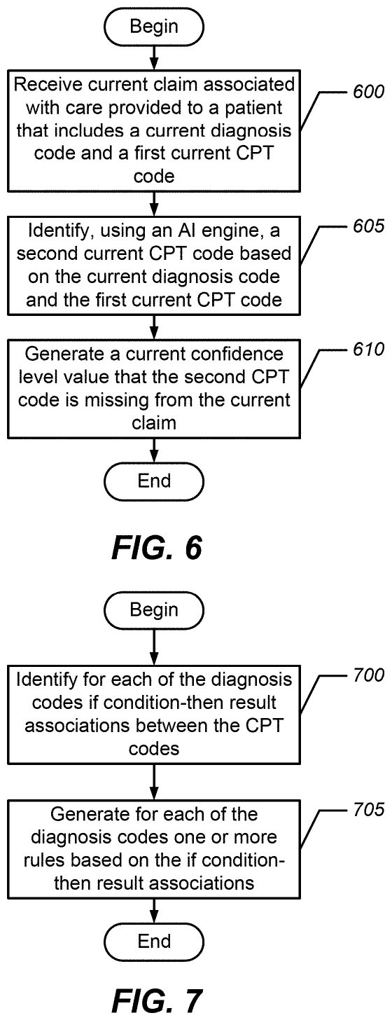

FIGS. 6-8 are flowcharts that that illustrate operations for capturing missing CPT codes from a claim in accordance with some embodiments of the inventive concept. Referring to FIG. 6, operations begin at block 600 where the CPT code capture system receives a current claim associated with care provided to a patient. The claim includes a current diagnosis code and a first current CPT code. An AI engine, such as the AI engine described above with respect to FIG. 2, may identify a second current CPT code based on the current diagnosis code and the first current CPT code at block 605. A current confidence level may be generated at block 610 that is indicative of the likelihood that the second CPT code is missing from the current claim.

Referring now to FIG. 7, the AI engine may be configured to use an ARL engine that has been trained using a plurality of historical claims associated with past care provided to a plurality of patients. These historical claims may include a plurality of diagnosis does and a plurality of CPT codes. For each of the diagnosis codes, if condition-then result associations are identified between the CPT codes at block 700. These associations may be between sets of the CPT codes where each set includes one or more CPT codes. At block 705, for each of the diagnosis codes, one or more rules are generated based on the if condition-then result associations identified at block 700.

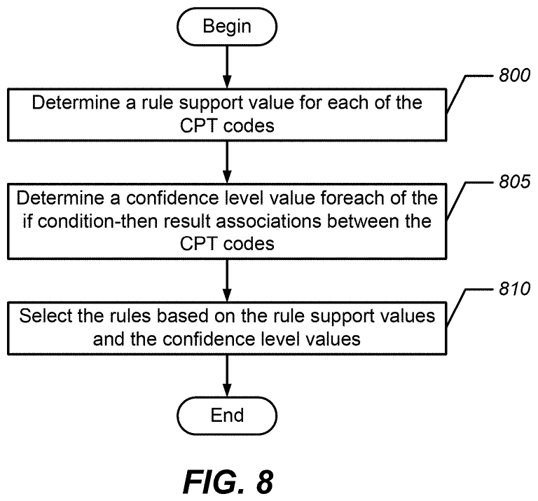

The rules that are generated may be a subset of the universe of possible rules to emphasize the rules that are more statistically significant. Referring to FIG. 8, a rule support value may be determined for each of the CPT codes or combination of codes used as an antecedent of consequent at block 800. The rule support value is indicative of the frequency of occurrence of the itemset, i.e., CPT code or combination of CPT codes, in the historical claim information dataset. A confidence level value for each of the if condition-then result associations between the itemsets is determined at block 805. The confidence level value indicates the likelihood of occurrence of the consequent given the antecedent. It may be determined as a proportion of occurrence on both antecedent and consequent over occurrence of the antecedent, i.e., a likelihood that a particular rule would have a true result. At block 810 the rules are selected based on the rule support values and the confidence level values.

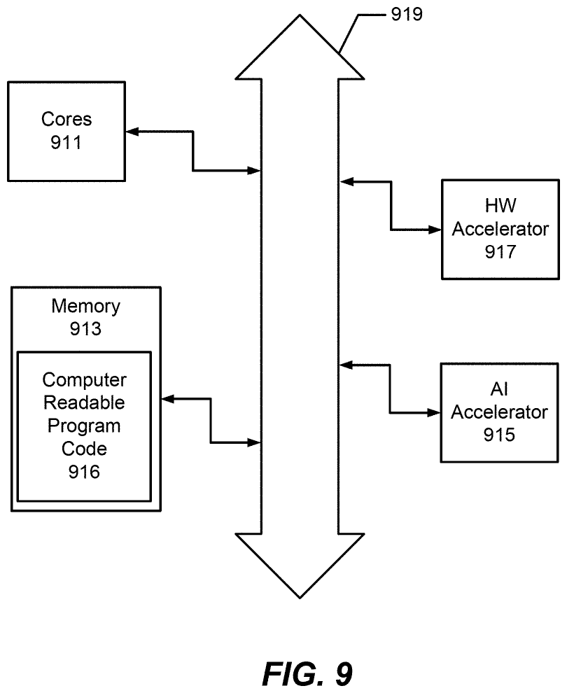

FIG. 9 is a block diagram of a data processing system that may be used to implement the CPT code capture server 130 of FIG. 1 in accordance with some embodiments of the inventive concept. As shown in FIG. 9, the data processing system may include at least one core 911, a memory 913, an artificial intelligence (AI) accelerator 915, and a hardware (HW) accelerator 917. The at least one core 911, the memory 913, the AI accelerator 915, and the HW accelerator 917 may communicate with each other through a bus 919.

The at least one core 911 may be configured to execute computer program instructions. For example, the at least one core 911 may execute an operating system and/or applications represented by the computer readable program code 916 stored in the memory 913. In some embodiments, the at least one core 911 may be configured to instruct the AI accelerator 915 and/or the HW accelerator 917 to perform operations by executing the instructions and obtain results of the operations from the AI accelerator 915 and/or the HW accelerator 917. In some embodiments, the at least one core 911 may be an ASIP customized for specific purposes and support a dedicated instruction set.

The memory 913 may have an arbitrary structure configured to store data. For example, the memory 913 may include a volatile memory device, such as dynamic random access memory (DRAM) and static RAM (SRAM), or include a non-volatile memory device, such as flash memory and resistive RAM (RRAM). The at least one core 911, the AI accelerator 915, and the HW accelerator 917 may store data in the memory 913 or read data from the memory 913 through the bus 919.

The AI accelerator 915 may refer to hardware designed for AI applications. In some embodiments, the AI accelerator 915 may include a ARL processing unit (NPU) configured to use machine learning models to analyze data for patterns or co-occurrences. The AI accelerator 915 may generate output data by processing input data provided from the at least one core 915 and/or the HW accelerator 917 and provide the output data to the at least one core 911 and/or the HW accelerator 917. In some embodiments, the AI accelerator 915 may be programmable and be programmed by the at least one core 911 and/or the HW accelerator 917. The HW accelerator 917 may include hardware designed to perform specific operations at high speed. The HW accelerator 917 may be programmable and be programmed by the at least one core 911.

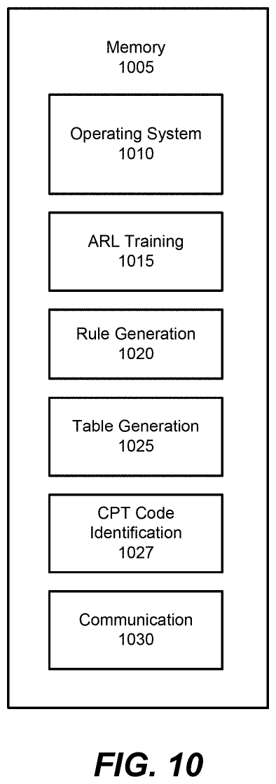

FIG. 10 illustrates a memory 1005 that may be used in embodiments of data processing systems, such as the CPT code capture server 130 of FIG. 1 and the data processing system of FIG. 9, respectively, to facilitate missing CPT code capture according to some embodiments of the inventive concept. The memory 1005 is representative of the one or more memory devices containing the software and data used for facilitating operations of the CPT code capture server 130 and the CPT code capture engine 135 as described herein. The memory 1005 may include, but is not limited to, the following types of devices: cache, ROM, PROM, EPROM, EEPROM, flash, SRAM, and DRAM. As shown in FIG. 10, the memory 1005 may contain six or more categories of software and/or data: an operating system 1010, an ARL training module 1015, a rule generation module 1020, a table generation module 1025, a CPT code identification module 1025, and a communication module 1030. In particular, the operating system 1010 may manage the data processing system's software and/or hardware resources and may coordinate execution of programs by the processor. The ARL training module 1015, the rule generation module 1020, the table generation module 1025, the CPT code identification module 1025, and the communication module 1030 may be configured to perform one or more operations described above with respect to the CPT code capture server 130 of FIG. 1. In some embodiments, the ARL training module 1015 may be configured to perform one or more of the operations described above with respect to the AI pattern detection module 205 and the support and confidence assessment module 210 of FIG. 2 and FIGS. 3-8. The rule generation module 1020 may be configured to perform one or more of the operations described above with respect to the rule generation module 215 of FIG. 2 and FIGS. 3-8. The table generation module 1025 may be configured to perform one or more operations described above with respect to the table generation module of FIG. 2 and FIGS. 3-8. The CPT code identification module 1027 may be configured to perform one or more operations described above with respect to the rule application module 225 of FIG. 3 and FIGS. 3-8. The communication module 1030 may be configured to facilitate communication between the CPT code capture server 130 and the CMS server 140a, insurance server 140b, and health care facility server 105.

Although FIGS. 10-11 illustrate hardware/software architectures that may be used in data processing systems, such as the CPT code capture server 130 of FIG. 1 and the data processing system of FIG. 9, respectively, in accordance with some embodiments of the inventive concept, it will be understood that the present invention is not limited to such a configuration but is intended to encompass any configuration capable of carrying out operations described herein.

Computer program code for carrying out operations of data processing systems discussed above with respect to FIGS. 1-10 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the CPT code capture server 130 of FIG. 1 and the data processing system of FIG. 9 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-9 may be used to facilitate missing CPT code capture from claims according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 1005 when coupled to a processor includes computer readable program code that, when executed by the processor, causes the processor to perform operations including one or more of the operations described herein with respect to FIGS. 1-8.

Some embodiments of the inventive concept may provide a CPT code capture system that may identify potential candidate CPT code(s) that may be missing from a current claim. Traditionally, coders and/or claim reviewers review claims generated for providers to look for errors and potential missing CPT codes. These coders and/or reviewers may develop expertise in certain medical areas corresponding to one or more diagnosis codes. As a result, these coders may develop experience in knowing what CPT codes are commonly used together for various treatment or care delivery regiments. Embodiments of the inventive concept may use an AI engine including ARL functionality to analyze large amounts of historical claim information to identify patterns between CPT codes for various diagnosis codes. These patterns can be embodied in rules, which can then be narrowed down to those rules that are the most statistically significant. The most statistically significant rules can be embodied as tables corresponding to the diagnosis codes, respectively. A new claim can then be analyzed through use of these tables to identify potentially missing CPT code(s). The system can notify a coder or reviewer of a claim with potentially missing CPT codes allowing the coder or reviewer to examine the claim in greater detail to determine if the claim needs to be amended in some way. By alerting coders or reviewers to those claims most likely to need modification, the time and labor associated with the claim review process may be reduced.

### Further Definitions and Embodiments

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
receiving a current claim associated with care provided to a patient by a provider, the claim including a current diagnosis code and a first current procedural terminology (CPT) code that are based on the care that was provided;
identifying, using an artificial intelligence (AI) engine, a second current CPT code based on the current diagnosis code and the first current CPT code; and
generating a current confidence level value that the second current CPT code is missing from the current claim.

2. The method of claim 1, further comprising:
generating a notification that the second current CPT code is missing from the current claim when the confidence level value exceeds a threshold value.

3. The method of claim 1, wherein identifying, using the (AI) engine, the second current CPT code based on the current diagnosis code and the first current CPT code comprises:
identifying, using an association rules learning (ARL) engine, the second current CPT code based on the current diagnosis code and the first current CPT code.

4. The method of claim 3, further comprising:
training the ARL engine using a plurality of historical claims associated with past care provided to a plurality of patients, the plurality of historical claims containing a plurality of diagnosis codes and a plurality of CPT codes.

5. The method of claim 4, wherein training the ARL engine comprises:
identifying, for each of the plurality of diagnosis codes, if condition-then result associations between ones of the plurality of CPT codes.

6. The method of claim 5, further comprising:
generating, for each of the plurality of diagnosis codes, at least one rule based on the if condition-then result associations between ones of the plurality of CPT codes;
wherein each of the at least one rule comprises an antecedent corresponding to the if condition, a consequent corresponding to the then result, a rule confidence level value that the consequent is found in combination with the antecedent in the plurality of historical claims for the respective one of the plurality of diagnosis codes.

7. The method of claim 6, further comprising:
determining a rule support value for each of the plurality of CPT codes in the plurality of historical claims based on a frequency of occurrence in the plurality of historical claims for each of the plurality of CPT codes;
determining a confidence level value for each of the if condition-then result associations between ones of the plurality of CPT codes based on a proportion of a number of times each of the at least one rule is found to be true relative to a frequency of a number of occurrences of the antecedent; and
selecting, for each of the plurality of diagnosis codes, the at least one rule based on the rule support values and the confidence level values.

8. The method of claim 6, wherein generating the current confidence level value that the second current CPT code is missing from the current claim comprises:
generating the current confidence value level as the rule confidence level value for one of the at least one rule corresponding to the current diagnosis code having the first CPT code as the antecedent and the second CPT code as the consequent.

9. The method of claim 6, wherein one of the at least one rule comprises one or more of the plurality of CPT codes as the antecedent and the one or more of the plurality of CPT codes as the consequent.

10. The method of claim 9, wherein the current claim includes a third CTP code;
wherein identifying, using the AI engine, the second CPT code comprises identifying, using the AI engine, the second CPT code based on the current diagnosis code, the first CPT code, and the third CPT code; and
wherein generating the current confidence level value comprises generating the current confidence value level as the rule confidence level value for one of the at least one rule corresponding to the current diagnosis code having the first CPT code and the third CPT code as the antecedent and the second CPT code as the consequent.

11. The method of claim 9, wherein identifying, using the AI engine, the second CPT code comprises identifying, using the AI engine, the second CPT code and a third CPT code based on the current diagnosis code and the first CPT code; and
wherein generating the current confidence level value comprises generating the current confidence value level as the rule confidence level value for one of the at least one rule corresponding to the current diagnosis code having the first CPT code as the antecedent and the second CPT code and the third CPT code as the consequent.

12. The method of claim 9, wherein the current claim includes a third CTP code;
wherein identifying, using the AI engine, the second CPT code comprises identifying, using the AI engine, the second CPT code and a fourth CPT code based on the current diagnosis code, the first CPT code, and the third CPT code; and
wherein generating the current confidence level value comprises generating the current confidence value level as the rule confidence level value for one of the at least one rule corresponding to the current diagnosis code having the first CPT code and the third CPT code as the antecedent and the second CPT code and the fourth CPT code as the consequent.

13. A system, comprising:
a processor; and
a memory coupled to the processor and comprising computer readable program code embodied in the memory that is executable by the processor to perform operations comprising:
receiving a current claim associated with care provided to a patient by a provider, the claim including a current diagnosis code and a first current procedural terminology (CPT) code that are based on the care that was provided;
identifying, using an artificial intelligence (AI) engine, a second current CPT code based on the current diagnosis code and the first current CPT code; and
generating a current confidence level value that the second current CPT code is missing from the current claim.

14. The system of claim 13, wherein identifying, using the (AI) engine, the second current CPT code based on the current diagnosis code and the first current CPT code comprises:
identifying, using an association rules learning (ARL) engine, the second current CPT code based on the current diagnosis code and the first current CPT code.

15. The system of claim 14, wherein the operations further comprise:
training the ARL engine using a plurality of historical claims associated with past care provided to a plurality of patients, the plurality of historical claims containing a plurality of diagnosis codes and a plurality of CPT codes.

16. The system of claim 15, wherein training the ARL engine comprises:
identifying, for each of the plurality of diagnosis codes, if condition-then result associations between ones of the plurality of CPT codes.

17. The system of claim 16, wherein the operations further comprise:
generating, for each of the plurality of diagnosis codes, at least one rule based on the if condition-then result associations between ones of the plurality of CPT codes;
wherein each of the at least one rule comprises an antecedent corresponding to the if condition, a consequent corresponding to the then result, a rule confidence level value that the consequent is found in combination with the antecedent in the plurality of historical claims for the respective one of the plurality of diagnosis codes.

18. The system of claim 17, wherein the operations further comprise:
determining a rule support value for each of the plurality of CPT codes in the plurality of historical claims based on a frequency of occurrence in the plurality of historical claims for each of the plurality of CPT codes;
determining a confidence level value for each of the if condition-then result associations between ones of the plurality of CPT codes based on a proportion of a number of times each of the at least one rule is found to be true relative to a frequency of a number of occurrences of the antecedent; and
selecting, for each of the plurality of diagnosis codes, the at least one rule based on the rule support values and the confidence level values.

19. A computer program product, comprising:
a non-transitory computer readable storage medium comprising computer readable program code embodied in the medium that is executable by a processor to perform operations comprising:
receiving a current claim associated with care provided to a patient by a provider, the claim including a current diagnosis code and a first current procedural terminology (CPT) code that are based on the care that was provided;
identifying, using an artificial intelligence (AI) engine, a second current CPT code based on the current diagnosis code and the first current CPT code; and
generating a current confidence level value that the second current CPT code is missing from the current claim.

20. The computer program product of claim 19, wherein identifying, using the (AI) engine, the second current CPT code based on the current diagnosis code and the first current CPT code comprises:
identifying, using an association rules learning (ARL) engine, the second current CPT code based on the current diagnosis code and the first current CPT code.
